SEQUENCE LISTING (4 sequences)

SEQ ID NO: 1 - protein, 140 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 2 - protein, 90 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNNC

SEQ ID NO: 3 - protein, 100 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKIWLYGNAETSR

SEQ ID NO: 4 - protein, 119 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

(19) United States
(12) Patent Application Publication
(10) Pub. No.: US 2017/0304400 A1
(43) Pub. Date: Oct. 26, 2017

(54) METHODS FOR PREVENTING FIBROSIS USING CXCR4 AND/OR CXCR7 BINDING AGENTS

(71) Applicants: ViCapsys.Inc, Cumming, GA (US); The General Hospital Corporation, Boston, MA (US)

(72) Inventors: Mark Poznansky, Charlestown, MA (US); James Markmann, Boston, MA (US); Timothy Brauns, Boston, MA (US); Marinko Sremac, Boston, MA (US); Nicolas Chronos, Cumming, GA (US); Suren Chavan, Cumming, GA (US)

(21) Appl. No.: 15/496,573

(22) Filed: Apr. 25, 2017

Related U.S. Application Data
(60) Provisional application No. 62/327,262, filed on Apr. 25, 2016.
(60) Provisional application No. 62/327,345, filed on Apr. 25, 2016.

(51) Int. Cl. A61K 38/19; A61K 9/48; A61K 9/00

(52) U.S. Cl. CPC A61K 38/195; A61K 9/0014; A61K 9/48

(57) ABSTRACT

This disclosure is directed to methods of treating a subject with a fibrotic or fibroproliferative disease, comprising administering to the subject a composition comprising an effective amount of an anti-fibrosis agent, e.g., a CXCR4 and/or CXCR7 binding agent, such as CXCL12.

### STATEMENT OF PRIORITY

This application claims the benefit of U.S. Provisional Application Ser. No. 62/327,262, filed Apr. 25, 2016, and U.S. Provisional Application Ser. No. 62/327,345, filed Apr. 25, 2016, the entire contents of which are incorporated by reference herein.

### STATEMENT REGARDING ELECTRONIC FILING OF A SEQUENCE LISTING

A Sequence Listing in ASCII text format, submitted under 37 C.F.R. §1.821, entitled 1416-5_ST25.txt, 4,651 bytes in size, generated on Apr. 24, 2017 and filed via EFS-Web, is provided in lieu of a paper copy. The Sequence Listing is incorporated herein by reference into the specification for its disclosures.

### FIELD OF THE INVENTION

This disclosure is directed to methods of treating a subject with a fibrotic or fibroproliferative disease, comprising administering to the subject a composition comprising an effective amount of an anti-fibrosis agent, e.g., a CXCR4 and/or CXCR7 binding agent, such as CXCL12 or a peptide derivative of CXCL12 or modified CXCL12 or a CXCR4 and/or CXCR7 agonist.

### BACKGROUND OF THE INVENTION

Fibrosis affects nearly all tissues and organ systems. Disorders are associated with and caused by excessive scarring and/or adhesions resulting from surgery, chemotherapeutic drug-induced fibrosis, radiation-induced fibrosis, and injuries and burns. Fibrotic tissue remodeling can also influence cancer metastasis and accelerate chronic graft rejection in transplant recipients. Fibrosis is also a component of the foreign body reaction to implanted devices. Diseases in which fibrosis is a major cause of morbidity and mortality include interstitial lung diseases, liver cirrhosis, liver fibrosis resulting from chronic hepatitis B or C infection, kidney disease, heart disease, and systemic sclerosis. Fibroproliferative disorders also include systemic and local scleroderma, keloids and hypertrophic scars, atherosclerosis, restenosis, and eye diseases including macular degeneration and retinal and vitreal retinopathy.

Fibrosis is central to the pathogenesis of many chronic lung disorders, including asthma, pneumoconioses, and many infections. The quintessential fibrotic lung diseases, however, are the fibrotic interstitial lung diseases, usual interstitial pneumonia (UIP) and fibrotic variant of non-specific interstitial pneumonia (NSIP). These illnesses are of unknown cause and are characterized by progressive lung fibrosis, typically culminating in respiratory failure and premature death. No treatment has been clearly effective in altering the clinical course of these diseases.

There is a long felt need in the art for methods of treating fibrotic diseases.

### SUMMARY OF THE INVENTION

Fibrotic diseases result from the undesired infiltration of fibrocytes to the site of the disease. Without being limited to any theory, this invention is predicated on the understanding that fibrocyte infiltration often arises as a consequence of prior and concurrent immune cell infiltration to the disease site. Fibrocytes may be recruited to the site by immune cells or by direct recruitment (e.g., via chemokine signaling). Thus, and without being bound by theory, the presence of immune cells at a site may be a precursor to recruitment, or fibrocytes may be recruited independently from and/or concurrently with immune cells. Based on that understanding, this invention provides a method for treating, preventing, and/or inhibiting fibrosis by inhibiting fibrocyte infiltration to the disease site either directly or indirectly. Specifically, this invention provides for the use of CXCR4 and/or CXCR7 binding agents at a site of fibrosis or at risk of fibrosis. CXCR4 and/or CXCR7 binding agents repel fibrocytes and/or immune cells and inflammatory cells that are understood to be essential components in the etiology of fibrosis.

CXCR4 and/or CXCR7 binding agents (e.g., CXCL12 (also called SDF-1) and other CXCR4- or CXCR7-binding molecules that activate the CXCL12/CXCR4 and/or CXCL12/CXCR7 pathway) repel effector T-cells and NK cells while recruiting immune-suppressive regulatory T-cells and M2 macrophages to an anatomic site. This action allows for a down-regulation of immune activity in the treated area thereby inhibiting fibrocyte migration to that area. In addition, fibrocytes themselves may express chemokine receptors (e.g., CXCR4) that are involved in the chemorepellant activity of CXCR4 and/or CXCR7 binding agents.

Certain chemokines have chemorepellant activity at high concentrations, and chemoattractant activity at low concentrations. In one embodiment, the CXCR4/CXCR7 binding agent is a CXCR4-binding molecule or a CXCR7-binding molecule. In one embodiment, the CXCR4 and/or CXCR7 binding agent is IL-8 or CXCL12 (also called SDF-la), or an active fragment thereof. In one embodiment, the CXCR4 and/or CXCR7 binding agent is CXCL12. Additional CXCR4 and/or CXCR7 binding agents are described, for example, in U.S. Pat. No. 6,448,054, which is incorporated herein by reference in its entirety. Without being bound by theory, it is believed that at least a subset of CXCR4 and/or CXCR7 binding agents inhibit or attenuate migration of immune cells to the site at risk of fibrosis thereby also attenuating the migration of fibrocytes.

In one aspect of the invention, this specification describes that the incorporation of the chemokine CXCL12 alone or within a coating or encapsulant at the time of insertion or surgery results in abrogation of intraperitoneal/intrapleural or intrapericardial inflammation and its sequelae including adhesions. This invention arises out of the recent studies in non-human primates in which clinical grade alginate microcapsules were placed in the intraperitoneal cavity of animals. At both 30 days and 100 days, alginate capsules containing CXCL12 remained free floating in the abdominal cavity without evidence of fibrosis or inflammation. This was unexpected and surprising, and in marked contrast to microcapsules without CXCL12 which were embedded in fibrous tissue in the mesentery and associated with peritoneal adhesions. This finding presented itself as a new potential approach to addressing the clinical issue of post-surgical adhesions or the generation of intraabdominal or other anatomic space fibrosis as a result of infection, interventions or the placement of devices. Blank capsules +/−CXCL12 and histopathology also demonstrated this marked difference in intraperitoneal inflammation post capsule implantation.

Direct application of a CXCR4 and/or CXCR7 binding agent, e.g., CXCL12, to the intraperitoneal cavity or other anatomic cavity post procedure either alone or in a gel, foam or on a platform matrix material, prevents/abrogates inflammation, fibrosis and/or adhesion formation. In some embodiments, microcapsules or nanoparticles serve as a release vehicle for the CXCR4 and/or CXCR7 binding agent, e.g., CXCL12. In some embodiments, the microcapsules or nanoparticles are biodegradable, e.g., made from alginate and/or hyaluronic acid, Without being bound by theory, it is believed that biodegradable microcapsules or nanoparticles are particularly useful for short-term use, where the disease or condition does not require long-term (e.g., several months or more) exposure to the CXCR4 and/or CXCR7 binding agent, and/or where the particles cannot be easily removed from the patient at a later time. The agents and particles may be used, for example, in an inhalant, e.g., with a carrier, or in a liquid, e.g., for instillation into an organ such as the bladder, or as a spray or ointment for topical use.

The application of CXCL12 into the intraperitoneal cavity in alginate capsules results in no adhesions following surgery or explantation of the capsules, whereas empty capsules without CXCL12 were associated with the formation of adhesions and fibrosis of inserted capsules.

This application is predicated on the understanding that CXCR4 and/or CXCR7 binding agents impart anti-fibrotic activity at the site of implantation, and that such is independent of encapsulation of the CXCR4 and/or CXCR7 binding agent. Accordingly, CXCR4 and/or CXCR7 binding agents administered directly to fibrotic lung tissue as an inhalant such as a nebulized inhalant will inhibit fibrosis including idiopathic pulmonary fibrosis. Likewise, CXCR4 and/or CXCR7 binding agents eluting from sutures will inhibit fibrosis in the surgical sites where the sutures are employed. Still further, sub-dermal application of CXCL12 can be used for fibrosis associated with acne scars and other fibrotic skin diseases. It can also be used to prevent or inhibit fibrosis due to chemotherapy or radiation and other insults such as burns and injuries.

In one aspect, this invention relates to a method for treating, preventing, or inhibiting a fibrotic lung disease in a subject in need thereof, the method comprising administering to the subject an effective amount of a CXCR4 and/or CXCR7 binding agent. In some embodiments, the CXCR4 and/or CXCR7 binding agent is administered directly to the lungs. In some embodiments, the CXCR4 and/or CXCR7 binding agent is associated with a particle that elutes the CXCR4 and/or CXCR7 binding agent at a desired elution rate.

In some embodiments, the fibrotic lung disease is selected from the group consisting of pulmonary fibrosis, fibrotic interstitial lung disease, interstitial pneumonia, fibrotic variant of non-specific interstitial pneumonia, cystic fibrosis, lung fibrosis, chronic obstructive pulmonary lung disease (COPD), and pulmonary arterial hypertension. In some embodiments, the fibrotic lung disease is pulmonary fibrosis.

In one aspect, this invention relates to a method of treating, preventing, or inhibiting fibrosis in a subject in need thereof comprising contacting a fibrotic site or a site at risk of fibrosis with an effective amount of a CXCR4 and/or CXCR7 binding agent. In some embodiments, the CXCR4 and/or CXCR7 binding agent is associated with and eluted from a particle. Such sites include surgical adhesions, acne scars, scars arising from disease states such a chicken pox, and the like. In some embodiments, the CXCR4 and/or CXCR7 binding agent is administered subdermally, e.g., via injection or topically.

In one aspect, this invention relates to a method of treating or inhibiting pulmonary fibrosis comprising inhibiting the migration of fibrocytes and/or activation of fibroblasts in a subject comprising contacting said fibrocytes and/or fibroblasts with a an effective amount of a composition comprising a CXCR4 and/or CXCR7 binding agent.

In one aspect, this invention relates to a method of treating, preventing, or inhibiting adhesions (e.g., post-surgical adhesions) in a subject in need thereof, the method comprising administering to the subject an effective amount of a CXCR4 and/or CXCR7 binding agent. In some embodiments, the CXCR4 and/or CXCR7 binding agent is administered during or immediately after surgery, e.g., intra-abdominal surgery or intra-thoracic surgery. In some embodiments, the CXCR4 and/or CXCR7 binding agent is administered generally to the thoracic or intraperitoneal region. In some embodiments, the CXCR4 and/or CXCR7 binding agent is administered locally, e.g., to an incision or wound site. In some embodiments, the CXCR4 and/or CXCR7 binding agent is administered during or immediately after insertion of a device to the subject, e.g., a medical device or implant. The agent may be coated on or be in the device or implant.

In some embodiments, the composition comprises immunorepellant, biodegradable, and non-cellular particles, wherein said particles are loaded with the CXCR4 and/or CXCR7 binding agent. In some embodiments, the particles encapsulate or are coated with the CXCR4 and/or CXCR7 binding agent.

In some embodiments, the CXCR4 and/or CXCR7 binding agent elutes from the particles in an amount sufficient to provide a chemorepellant environment at the site of implantation or injection for a period of at least one month after implantation or injection. In some embodiments, the particles have an average diameter of between about 1 micron to about 20 microns.

In some embodiments and depending on the disease or condition to be treated, the CXCR4 and/or CXCR7 binding agent may be administered as a composition which can be formulated for inhalation, spray, injection, topical, or incorporated into sutures, etc. For example, for lung fibrosis, the composition may be an inhalable formulation. For prevention/inhibition of intraperitoneal adhesions and scarring, the composition may be a sprayable formulation or a topical formulation.

In some embodiments, the CXCR4 and/or CXCR7 binding agent may be a CXCR4-binding molecule or a CXCR7-binding molecule that activates the CXCL12/CXCR4 and/or CXCL12/CXCR7 pathway. In some embodiments, the CXCR4-binding molecule is CXCL12 or an active fragment or analog thereof.

In some embodiments, the CXCR4 and/or CXCR7 binding agent may be administered in combination with a co-agent, e.g., an additional therapeutic agent. In some embodiments, the co-agent is an antibiotic, a corticosteroid, an immunosuppressive agent, an anticoagulant, a diuretic, a cardiac glycoside, a calcium channel blocker, a vasodilator, a prostacyclin analogue, an endothelin antagonist, a phosphodiesterase inhibitor, a beta-2 agonist, an antimuscarinic agent, an endopeptidase inhibitor, a lipid lowering agent or a thromboxane inhibitor, or combinations thereof.

### BRIEF DESCRIPTION OF THE DRAWINGS

FIG. 1 shows the parameters of CXCL12⁻ microbeads.

FIG. 2 shows the parameters of CXCL12⁺ microbeads.

FIGS. 3A-3B show images of the 30-day explant procedure.

FIGS. 4A-4B show hematoxylin and eosin (H&E) stained sections of (A) CXCL2⁻ and (B) CXCL12⁺ embedded microbeads. Top set magnification at 10×; bottom set magnification at 20×.

FIG. 5 shows images of CXCL12− (top) and CXCL12+ microbeads

FIG. 6 shows H&E stained sections of embedded microbeads. Left: 90 days (CXCL12⁻ on top; CXCL12⁺ on bottom). Right: 180 days (all images CXCL12⁺). Magnification at 300×.

FIG. 7 shows plasma cytokine levels in CXCL12⁺ and CXCL12⁻ non-human primates (NHPs). The hatched lines show average control blood cytokine levels in healthy non-transplanted NHPs from published studies.

FIG. 8 shows images of CXCL12⁺ (left) and CXCL12⁻ (right) xenoislet implants. Arrows show fibrotic adhesions.

### DETAILED DESCRIPTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this invention belongs.

All publications, patent applications, patents, patent publications and other references cited herein are incorporated by reference in their entireties for the teachings relevant to the sentence and/or paragraph in which the reference is presented.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

In this specification and in the claims that follow, reference will be made to a number of terms that shall be defined to have the following meanings:

As used herein, the singular forms “a”, “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely examples and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “fibrotic” disease or a “fibroproliferative” disease refers to a disease characterized by scar formation and/or the over production of extracellular matrix by connective tissue. Fibrotic disease occurs as a result of tissue damage. It can occur in virtually every organ of the body. Examples of fibrotic or fibroproliferative diseases include, but are not limited to, pulmonary fibrosis, idiopathic pulmonary fibrosis, fibrotic interstitial lung disease, interstitial pneumonia, fibrotic variant of non-specific interstitial pneumonia, cystic fibrosis, lung fibrosis, silicosis, asbestosis, asthma, chronic obstructive pulmonary lung disease (COPD), pulmonary arterial hypertension, liver fibrosis, liver cirrhosis, renal fibrosis, glomerulosclerosis, diabetic nephropathy, heart disease, fibrotic valvular heart disease, systemic fibrosis, rheumatoid arthritis, excessive scarring resulting from surgery or other injury, adhesions, chemotherapeutic drug-induced fibrosis, radiation-induced fibrosis, macular degeneration, retinal and vitreal retinopathy, atherosclerosis, and restenosis. Fibrotic disease or disorder, fibroproliferative disease or disorder and fibrosis are used interchangeably herein.

The terms “patient,” “subject,” “individual,” and the like are used interchangeably herein, and refer to any animal, or cells thereof whether in vitro or in situ, amenable to the methods described herein. In one embodiment, the patient, subject, or individual is a mammal. In some embodiments, the mammal is a mouse, a rat, a guinea pig, a non-human primate, a dog, a cat, or a domesticated animal (e.g., horse, cow, pig, goat, sheep). In certain embodiments, the patient, subject or individual is a human.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectable level or activity (at most, an insignificant amount, e.g., less than about 10% or even 5%).

The term “contact” or grammatical variations thereof as used with respect to a polypeptide and a receptor, refers to bringing the polypeptide and the receptor in sufficiently close proximity to each other for one to exert a biological effect on the other. In some embodiments, the term contact means binding of the polypeptide to the receptor.

The term “treat,” “treating,” or “treatment” (and grammatical variations thereof) covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

The term “decreasing risk” refers to lowering the likelihood of establishing a disease, disorder, or condition and/or decreasing the severity or extent of a disease, disorder or condition if it is established.

The term “administering” or “administration” of an agent or drug to a subject includes any route of introducing or delivering to a subject a compound to perform its intended function. Administration can be carried out by any suitable route, including orally, intranasally, by inhalation, or parenterally (intravenously, intramuscularly, intraperitoneally, subdermally, or subcutaneously). Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved.

The term “therapeutic” as used herein means a treatment and/or prophylaxis. A therapeutic effect is obtained by suppression, remission, or eradication of a disease state.

The term “therapeutically effective amount” or “effective amount” refers to an amount of the agent that, when administered, is sufficient to cause the desired effect. For example, an effective amount of CXCL12 may be an amount sufficient to have a chemorepellant effect on an immune cell. The therapeutically effective amount of the agent will vary depending on the disease or condition being treated and its severity as well as the age, weight, etc., of the subject to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

“Cytokine” is a generic term for non-antibody, soluble proteins which are released from one cell subpopulation and which act as intercellular mediators, for example, in the generation or regulation of an immune response. See Human Cytokines: Handbook for Basic & Clinical Research (Aggrawal, et al., eds., Blackwell Scientific, Boston, Mass. 1991) (which is hereby incorporated by reference in its entirety for all purposes).

By “chemorepellant activity” it is meant the ability of an agent to repel (or chemorepel) a eukaryotic cell with migratory capacity (i.e., a cell that can move away from a repellant stimulus). Accordingly, an agent with chemorepellant activity is a “chemorepellant agent.” Such activity can be detected using any of a variety of systems well known in the art (see, e.g., U.S. Pat. No. 5,514,555 and U.S. Patent Application Publication No. 2008/0300165, each of which is incorporated by reference herein in its entirety). A preferred system for use herein is described in U.S. Pat. No. 6,448,054, which is incorporated herein by reference in its entirety.

A compound exhibiting chemorepellant activity at a given concentration is referred to as a “chemorepellant agent.” As these chemorepellant agents inhibit fibrosis, these agents are sometimes referred to herein as “anti-fibrotic agents.”

The term “chemorepellant effect” refers to the chemorepellant effect of a chemokine or other chemorepellant agent. Usually, the chemorepellant effect is present in an area around a cell which secretes the chemokine (e.g., a tumor cell) wherein the concentration of the chemokine is sufficient to provide the chemorepellant effect. Some chemokines, including interleukin 8 (IL-8) and CXCL12, may exert chemorepellant activity at high concentrations (e.g., over about 100 nM), whereas lower concentrations exhibit no chemorepellant effect and may even be chemoattractant. As described herein, the chemorepellant effect may be in an area surrounding administration of the chemorepellant agent.

The term “chemorepellant environment” refers to an area that has a sufficient level of chemorepellant agent to exert a chemorepellant effect.

The term “CXCR4 and/or CXCR7 binding agent” refers to a compound or molecule that binds to CXCR4 and/or CXCR7 and activates a CXCL12/CXCR4 and/or CXCL12/CXCR7 pathway thereby causing a chemorepellant or immunorepellant effect.

“Immune cells” as used herein are cells of hematopoietic origin that are involved in the specific recognition of antigens. Immune cells include antigen presenting cells (APCs), such as dendritic cells or macrophages, B cells, natural killer cells, T cells, etc.

As used herein, the term “non-cellular” in reference to a particle as described herein indicates that the particle does not contain one or more cells, e.g., one or more cells are not encapsulated within the particle.

As used herein, the term “immunorepellant” refers to an ability to repel immune or other cells from a given site.

I. Compositions Comprising CXCR4 and/or CXCR7-Binding Molecule

In one aspect, this invention is directed to a composition comprising a CXCR4 and/or CXCR7 binding agent. In one embodiment, the CXCR4 and/or CXCR7 binding agent is a CXCR4-binding molecule or a CXCR7-binding molecule that activates the CXCL12/CXCR4 and/or CXCL12/CXCR7 pathway. CXCR4 and/or CXCR7 binding agents are described, for example, in U.S. Pat. No. 6,448,054, which is incorporated herein by reference in its entirety. CXCR4-binding molecules include, but are not limited to, CXCL12 or active fragments or derivatives or analogs thereof, including protease-resistant derivatives, an agonistic antibody to CXCR4, or any other ligand for CXCR4. CXCR4 agonists are disclosed, for example, in US Publication Nos. 2017/0079971, 2016/0228413, 2015/0157630, 2015/0038509, 2013/0324552, 2013/0210709, 2013/0079292, 2013/0035347, 2013/0005944, and 2012/0301427, each incorporated by reference herein in its entirety. CXCR7-binding molecules include, but are not limited to, CXCL12 or active fragments or derivatives or analogs thereof, including protease-resistant derivatives, an agonistic antibody to CXCR7, or any other ligand for CXCR7. CXCR7 agonists are disclosed, for example, in US Publication Nos. 2016/0107997, 2015/0307556, 2013/0345199, 2013/0225506, 2013/0023483, 2009/0098091, and 2007/0160574, each incorporated by reference herein in its entirety.

In one embodiments, the CXCR4-binding molecule is CXCL12 (CXCL12 polypeptide). CXCL12 or CXCL12 polypeptide refers to a protein or fragment thereof that binds a CXCL12 specific antibody and that has chemorepellant or immunorepellant activity. Chemorepellant activity is determined by assaying the direction of T cell migration (e.g., toward or away from an agent of interest). See, e.g., Poznansky et al., Nature Medicine 2000, 6:543-8.

CXCL12 polypeptides are known in the art. See, e.g., Poznansky et al., Nature Medicine 2000, 6:543-8, which is incorporated herein in its entirety. In one embodiment, a CXCL12 polypeptide has at least about 85%, 90%, 95%, or 100% amino acid sequence identity to NP_001029058 and has chemokine activity. Examples of SDF-1 (CXCL12) isoforms can be found in PCT Publication No. WO 2015/069256, which is incorporated herein by reference in its entirety. These include SDF-1 alpha (Accession No. NP_954637), SDF-1 beta (Accession No. P48061), SDF-1 gamma (Accession No. NP_001029058), SDF-1 delta (MNAKVVVVLVLVLTALCLSDGKPVSLSYRCPCRFFESHVARANVKHLKILNTPNCALQ IVARLKNNNRQVCIDPKLKWIQEYLEKALNNLISAAPAGKRVIAGARALHPSPPRACPTA RALCEIRLWPPPEWSWPSPGDV (SEQ ID NO:1)), SDF-1 epsilon (MNAKVVVVLVLVLTALCLSDGKPVSLSYRCPCRFFESHVARANVKHLKILNTPNCALQ IVARLKNNNRQVCIDPKLKWIQEYILEKALNNC (SEQ ID NO:2)), and SDF-1 phi (MNAKVVVVLVLVLTALCLSDGKPVSLSYRCPCRFFESHVARANVKHLKILNTPNCALQ IVARLKNNNRQVCIDPKLKWIQEYLEKALNKIWLYGNAETSR (SEQ ID NO:3)). In another embodiment, the sequence of the CXCL12/SDF-1 polypeptide is MNAKVVVVLVLVLTALCLSDGKPVSLSYRCPCRFFESHVARANVKHLKILNTPNCALQI VARLKNNRQVCIDPKLKWIQEYLEKALNKGRREEKVGKKEKIGKKKRQKKRKAAQK RKN (SEQ ID NO:4). In one embodiment, a CXCL12 polypeptide has at least about 85%, 90%, 95%, or 100% amino acid sequence identity to a sequence described herein and has chemokine or anti-fibrosis activity.

In one aspect, this invention relates to a composition suitable for implantation or injection into a patient, wherein the composition comprises an effective amount of an anti-fibrosis agent, e.g., a CXCR4 and/or CXCR7 binding agent. In some embodiments, the composition comprises immunorepellant, biodegradable, and non-cellular particles, wherein said particles are loaded with the CXCR4 and/or CXCR7 binding agent. In some embodiments, the particles encapsulate or are coated with the CXCR4 and/or CXCR7 binding agent.

In one aspect, the invention is directed to a particle or particles that is/are loaded with a CXCR4 and/or CXCR7 binding agent. Preferably, the CXCR4 and/or CXCR7 binding agent elutes from the particles in an amount sufficient to provide a chemorepellant environment at the site of implantation or injection of the particle(s). In one embodiment, the chemorepellant environment is maintained at the site of implantation or injection for a period of at least one month after implantation or injection, e.g., at least 2, 3, 4, 5, or 6 months.

In some embodiments, the CXCR4 and/or CXCR7 binding agent elutes from the particles in an effective amount, e.g., an amount sufficient to provide a chemorepellant environment at the site of implantation or injection for a period of at least one month after implantation or injection, e.g., at least 2, 3, 4, 5, or 6 months. In some embodiments, the particles have an average diameter of between about 1 micron to about 20 microns, e.g., 1, 2, 3, 4, 5, 6, 7, 8, 9, 10, 11, 12, 13, 14, 15, 16, 17, 18, 19, or 20 microns or any range therein. In certain embodiments, the CXCR4 and/or CXCR7 binding agent is CXCL12.

In one embodiment, the composition does not comprise an alginate particle.

In some embodiments, the effective amount is between about 0.01 μg/mL to about 500 μg/mL, e.g., in a composition to be administered to a subject and/or after administration to a subject. In some embodiments, the effective amount is between about 0.1 μg/mL to about 50 μg/mL. In some embodiments, the effective amount is between about 0.5 μg/mL to about 5 μg/mL. In some embodiments, the effective amount is about 1 μg/mL. Contemplated values include any range between any of the above two values, or any value or subrange there between.

In some embodiments, about 5 mL to about 20 ml, of a pharmaceutical composition comprising the agent at a concentration between about 0.5 μg/mL and about 500 μg/mL is nebulized (or otherwise aerosolized), e.g., for application to the lungs. In some embodiments, the agent is present at a concentration between about 0.1 μg/mL to about 50 μg/mL. In some embodiments, the agent is present at a concentration between about 0.5 μg/mL to about 5 μg/mL. Contemplated values include any range between any of the above two values, or any value or subrange there between.

In some embodiments, a solution comprising between about 0.1 μg/mL and about 500 μg/mL of the agent is applied to a lesion or area. In some embodiments, the agent is present at a concentration between about 0.1 μg/mL to about 50 μg/mL. In some embodiments, the agent is present at a concentration between about 0.5 μg/mL to about 5 μg/mL. In some embodiments, the effective amount is between about 0.01 μg/mL to about 500 μg/ml per cm² of the region to be treated, e.g., between about 0.5 μg/mL to about 5 μg/mL per cm². Contemplated values include any range between any of the above two values, or any value or subrange there between.

In some embodiments, a solution comprising particles that elute the CXCR4 and/or CXCR7 binding agent to a concentration of between about 0.5 μg/mL and about 500 μg/mL is applied to a lesion or area. In some embodiments, the agent is eluted to a concentration between about 0.1 μg/mL to about 50 μg/mL. In some embodiments, the agent is eluted to a concentration between about 0.5 μg/mL to about 5 μg/mL. In some embodiments, the effective amount is between about 0.01 μg/mL to about 500 μg/ml per cm² of the region to be treated, e.g., between about 0.5 μg/mL to about 5 μg/mL per cm². Contemplated values include any range between any of the above two values, or any value or subrange there between.

The amount of agent administered depends on the weight, condition, and/or age of the subject, as well as the attending clinician's evaluation. The exact amount administered can vary within the ranges provided.

In one aspect, the composition is an injectable formulation, a sprayable formulation for use in subdermal applications such as a surgical field, or an inhalable formulation. The type of formulation is dependent, at least in part, on the intended use of the formulation.

In one aspect, the composition comprises particles. The particles may comprise a biocompatible material. The type of biocompatible material depends on the intended use. For example, the biocompatible material may be biodegradable or non-biodegradable. Without being bound by theory, it is believed that a biodegradable particle is preferred, for example, where the CXCR4 and/or CXCR7 binding agent is required at the site of implantation for a short period of time (e.g., hours to days or a week); where the particle cannot easily be removed from the implantation site; and/or where particles may cause damage or injury if left in place for a long period of time. In contrast, and without being bound by theory, it is believed that a non-biodegradable nanoparticle is preferred, for example, where the CXCR4 and/or CXCR7 binding agent is required at the site of implantation for a long period of time (e.g., weeks to months or longer), and/or the particle can be easily removed from the implantation site.

In one embodiment, the biocompatible material is a biocompatible polymer. The biocompatible polymer can be carbohydrate-based, protein-based, and/or synthetic, e.g., PLA. Biocompatable materials suitable for use in matrices include, but are not limited to, poly-dimethyl-siloxane (PDMS), poly-glycerolsebacate (PGS), polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, nondegradable polyurethanes, polystyrenes, polyvinyl chloride, polyvinyl fluoride, polyvinyl imidazole, chlorosulphonated polyolefins, polyethylene oxide, polyvinyl alcohol, nylon silicon, poly(styrene-block-butadiene), polynorbomene, and hydrogels. Other suitable polymers can be obtained by reference to The Polymer Handbook, 3rd edition (Wiley, N.Y., 1989). Combinations of these polymers may also be used. In one embodiment, the biocompatible polymer is alginate. In one embodiment, the biocompatible polymer is not alginate.

In one embodiment, the particle is retrievable. For example, the particle may comprise a metal that allows for magnetic retrieval of the particle(s) from the subject.

In one embodiment, the particle is visualizable. That is, a clinician can visualize the particle(s) within the subject in a non-invasive manner. For example, the particle may comprise a metal or other element that can be visualized non-invasively (e.g., by X-ray, MRI, CAT-scan, etc.). In some embodiments, the particle comprises a fluorescent marker or radioactive label that can be visualized non-invasively.

In one aspect, the anti-fibrosis effect (i.e., the ability of the composition to prevent fibrosis) in an area surrounding the particle(s) is maintained for at least 1 day to at least 1 month or more. In some embodiments, the anti-fibrosis effect in an area surrounding the particle(s) is maintained for at least one day, at least 2 days, at least 3 days, at least 4 days, at least 5 days, or at least 6 days. In some embodiments, the anti-fibrosis effect in an area surrounding the particle(s) is maintained for at least one week, at least two weeks, at least 3 weeks, at least 4 weeks, or at least 5 weeks. In some embodiments, the anti-fibrosis effect in an area surrounding the particle(s) is maintained for at least one month, at least 2 months, at least 3 months, at least 4 months, at least 5 months, at least 6 months, at least 7 months, at least 8 months, at least 9 months, at least 10 months, at least 11 months, or at least one year.

In some embodiments, the anti-fibrosis agent, e.g., a CXCR4 and/or CXCR7 binding agent, is administered in combination with a co-agent, e.g., an additional therapeutic agent. In some embodiments, the co-agent is an antibiotic, a corticosteroid, an immunosuppressive agent, an anticoagulant, a diuretic, a cardiac glycoside, a calcium channel blocker, a vasodilator, a prostacyclin analogue, an endothelin antagonist, a phosphodiesterase inhibitor, a beta-2 agonist, an antimuscarinic agent, an endopeptidase inhibitor, a lipid lowering agent or a thromboxane inhibitor, or combinations thereof. The co-agent may be a part of the composition with the anti-fibrosis agent, e.g., a CXCR4 and/or CXCR7 binding agent, or may be administered as a separate composition.

### II. Method of Treating and Preventing Fibrotic or Fibroproliferative Diseases

In one embodiment, the anti-fibrosis agent, e.g., a CXCR4 and/or CXCR7 binding agent, or composition as described herein can be used to treat, prevent, inhibit, or lower the risk of fibrotic or fibroproliferative disease or condition in a subject in need thereof. In one embodiment, the fibrotic or fibroproliferative disease is pulmonary fibrosis. In one embodiment, the fibrotic or fibroproliferative condition is adhesion formation (e.g., post-surgical adhesion).

In some embodiments, the anti-fibrosis agent, e.g., a CXCR4 and/or CXCR7 binding agent, or composition is administered to the patient to prevent formation of adhesions at a site of surgery or other injury. In some embodiments, the anti-fibrosis agent or composition is administered to the patient to prevent formation of adhesions at a site of implantation, e.g., of a device. In some embodiments, the implanted device is coated or sprayed with the anti-fibrosis agent or composition prior to, during, or after implantation. In some embodiments, the composition is administered directly to the patient's tissue(s) (e.g., by spray, topical administration (e.g., direct application to internal tissues, including the peritoneal cavity), injection, and the like). In some embodiments, the anti-fibrosis agent or composition is administered at the time of implantation or surgery. In some embodiments, the anti-fibrosis agent or composition is administered at the time of or soon after injury.

In one aspect, the invention is directed to the incorporation of the chemokine (e.g., CXCL12) alone or within a coating or encapsulant at the time of insertion or surgery resulting in abrogation of intraperitoneal/intrapleural or intrapericardial inflammation and its sequelae including adhesions.

In one aspect, this invention relates to a method for treating a fibrotic lung disease in a subject in need thereof, the method comprising administering to the patient an effective amount (e.g., a therapeutically or prophylactically effective amount) of an anti-fibrosis agent, e.g., a CXCR4 and/or CXCR7 binding agent, or composition as described herein. In some embodiments, the composition comprises particles which elute a CXCR4 and/or CXCR7 binding agent.

In one aspect, this invention relates to a method of treating or inhibiting pulmonary fibrosis comprising inhibiting the migration of fibrocytes and/or activation of fibroblasts in a subject comprising contacting said fibrocytes and/or fibroblasts with an effective amount (e.g., a therapeutically or prophylactically effective amount) of an anti-fibrosis agent, e.g., a CXCR4 and/or CXCR7 binding agent, or composition as described herein. In some embodiments, the composition comprises particles which elute a CXCR4 and/or CXCR7 binding agent.

In some embodiments, the fibrotic lung disease may be selected from pulmonary fibrosis, fibrotic interstitial lung disease, interstitial pneumonia, fibrotic variant of non-specific interstitial pneumonia, cystic fibrosis, lung fibrosis, chronic obstructive pulmonary lung disease (COPD), or pulmonary arterial hypertension.

Inflammation is the coordinated response to tissue injury or infection. Inflammation begins with the local release of chemotactic factors, platelet activation, and initiation of the coagulation and complement pathways. These events stimulate the local endothelium, promoting the extravasation of neutrophils and monocytes. The second phase of inflammation is characterized by the influx into the tissue of cells of the adaptive immune system, including lymphocytes. The subsequent resolution phase, when apoptosis of the excess leukocytes and engulfment by tissue macrophages takes place, is also characterized by repair of tissue damage by stromal cells, such as fibroblasts.

In such repair mechanisms, local quiescent fibroblasts migrate into the affected area, produce extracellular matrix proteins, and promote wound contraction or fibrosis. It has also been suggested that circulating fibroblast precursor cells, fibrocytes, which are present within the blood migrate to the sites of injury or fibrosis, where they differentiate and mediate tissue repair and other fibrotic responses. Fibrocytes differentiate from a CD14⁺ peripheral blood monocyte precursor population and express markers of both hematopoietic cells (CD45, MHC class II, CD34) and stromal cells (collagen types I and III and fibronectin). Mature fibrocytes rapidly enter sites of tissue injury where they secrete inflammatory cytokines, as well as extracellular matrix proteins, other cytokines and pro-angiogenic molecules, which may result in fibrosis.

Fibrocyte differentiation is associated with a variety of fibrotic diseases including but not limited to scleroderma, keloid scarring, rheumatoid arthritis, lupus, nephrogenic fibrosing dermopathy, and idiopathic pulmonary fibrosis. They play a role in the formation of fibrotic lesions after Schistosoma japonicum infection in mice and are also implicated in fibrosis associated with autoimmune diseases. Fibrocytes have also been implicated in pathogenic fibrosis associated with radiation damage, Lyme disease and pulmonary fibrosis, as well as stromal remodeling in pancreatitis and stromal fibrosis, whereas lack of such fibrocytes is associated with pancreatic tumors and adenocarcinomas. Fibrosis additionally occurs in asthma and possibly other pulmonary diseases such as chronic obstructive pulmonary disease when fibrocytes undergo further differentiation into myofibroblasts.

A specific set of diseases that are known to involve unchecked fibrotic response are idiopathic interstitial pneumonias, a diverse group of chronic pulmonary diseases characterized by varying levels of pulmonary fibrosis. The major factors driving the dominant pulmonary fibrotic response associated with various histologically distinct forms of idiopathic interstitial pneumonia (IIP) remains poorly defined, thereby contributing to the lack of effective clinical treatments for these diseases (Green, Overview of pulmonary fibrosis. Chest 2002; 122 (suppl. 6):334S-9S). Although many of these diseases exhibit a fibroproliferative response in the alveolar microenvironment leading to respiratory impairment, the degree of fibrotic change varies considerably among them (Nicholason, Am. J. Resp. Crit. Car Med/2000:162:2213-7; Chapman, J. Clin., Invest., 2004; 113:148-57). Equally perplexing is the clear demonstration that anti-inflammatory agents provide therapeutic benefit in less severe forms of IIP, such as non-specific interstitial pneumonia (NSIP) and respiratory bronchiolitis/interstitial lung disease (RBILD), but they often fail to prevent respiratory failure in patients with the most severe and deadly form of IIP—usual interstitial pneumonia (UIP) (Flaherty et al., Am. J. Med., 110:278-282 (2001); Lynch et al., Curr. Opin. Pulm. Med., 7:298-308 (2001); Flaherty et al., Thorax, 58:143-148 (2003)).

The fibrosis diseases treatable by an anti-fibrosis agent, e.g., a CXCR4 and/or CXCR7 binding agent, or composition as described herein may be any fibrosing disorder, including, but not limited to one that is selected from the group consisting of pulmonary fibrosis, chronic obstructive pulmonary disease, hepatic fibrosis, rheumatoid arthritis, chronic renal disease, hypersensitivity pneumonitis, respiratory bronchiolitis/interstitial lung disease, Schistosoma mansoni infection, primary pulmonary hypertension (prevention of the formation of the plexiform lesion) herpes virus associated-diseases, which include lung and dermatological manifestations, keloid scarring, lupus, nephrogenic fibrosing dermopathy, fibrosing lesions associated with Schistosoma japonicum infection, autoimmune diseases, pathogenic fibrosis, Lyme disease, stromal remodeling in pancreatitis and stromal fibrosis, uterine fibroids, ovarian fibrosis, corneal fibrosis, congestive heart failure and other post-ischemic conditions, post-surgical scarring including abdominal adhesions, wide angle glaucoma trabeculotomy, or any combination thereof.

Pulmonary fibrosis is a common consequence and often a central feature of many lung diseases. In some disorders, fibrosis develops focally and to a limited degree. For example, in asthma and chronic obstructive pulmonary disease fibrotic changes occur around conducting airways where scarring may be important to the pathophysiology.

The diagnosis of these conditions can usually be made by careful history, physical examination, chest radiography, including a high resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

Idiopathic pulmonary fibrosis (IPF) is clinically a restrictive lung disease that characteristically progresses relentlessly to death from respiratory failure. Median survival of newly diagnosed patients with IPF is about 3 years. The quality of life for IPF patients is also poor. Despite this, there has been remarkably little progress in development and/or assessment of therapeutic strategies for IPF.

Pulmonary function tests may be employed to detect physiological changes associated with the presence of pulmonary disease. Pulmonary function tests performed in a clinical setting may be used to evaluate lung mechanics, gas exchange, pulmonary blood flow, and blood gases and pH. They are used to evaluate patients in the diagnosis of pulmonary disease, assessment of disease development, or evaluation of the risk of pulmonary complications from surgery.

Pulmonary function tests are used to indicate a battery of studies or maneuvers that may be performed using standardized equipment to measure lung function. Pulmonary function tests include simple screening spirometry, formal lung volume measurement, diffusing capacity for carbon monoxide, and arterial blood gases.

The pulmonary function tests may obtain such values as FEV (forced expiratory volume), FVC (forced vital capacity), FEF_{25%-75%} (forced expiratory flow rate), PEFR (peak expiratory flow rate), FRC (functional residual capacity), RV (residual volume), TLC (total lung capacity), and/or flow/volume loops. FEV measures the volume of air exhaled over a predetermined period of time by a forced expiration immediately after a full inspiration. FVC measures the total volume of air exhaled immediately after a full inspiration. FEF_{25%-75%} measures the rate of air flow during a forced expiration divided by the time in seconds for the middle half of expired volume. PEFR measures the maximum flow rate during a forced exhale starting from full inspiration. FRC is the volume of air remaining in the lungs after a full expiration. RV is the FRC minus the expiratory reserve volume. TLC is the total volume in the lungs at the end of a full inspiration. Flow/volume loops are graphical presentations of the percent of total volume expired (on the independent axis) versus the flow rate during a forced expiratory maneuver. Normal values and lower limits of normal can be determined as defined by Hankinson et al. (the National Health and Nutrition Examination Survey [NHANES] III predicted set).

Targeting of a particular lung region depends on a number of factors, for example particle and breathing parameters, the mode of inhalation (steady state, single breath, and bolus inhalation), and the composition of the gas in which particles are inhaled. In one aspect, the material and size of the particles is dependent on the lung region to be targeted. Particle behavior in the human respiratory tract is well studied and understood, as set forth in Heyder, Proceedings of the American Thoracic Society, Vol. 1, NINETEENTH TRANSATLANTIC AIRWAY CONFERENCE (2004), pp. 315-320; and Tena and Clara, Arch Bronconeumol. 2012; 48(7):240-246, each of which is incorporated herein by reference in its entirety. In one embodiment, the particles are made of a hydrophobic material. In one embodiment, the particles are made of a hydrophilic material. In some embodiments, the particles are biodegradable. In certain embodiments, the particles are not alginate particles. Liquid particles (e.g., droplets) are also contemplated.

It can generally be considered that particles with a mass median aerodynamic diameter (MMAD) (diameter of a particle of mass equal to the average particle diameter of a population, i.e., the diameter of a particle in which 50% of the aerosol mass is greater and the other 50% is smaller) greater than 10 μm are deposited in the oropharynx, those between 5 and 10 μm are deposited in the central airways and those from 0.5 to 5 μm are deposited in the small airways and alveoli. Therefore, for topical respiratory treatment it is best to use particles with a MMAD between 0.5 and 5 μm. In one embodiment, the MMAD of the particles is greater than about 10 μm. In one embodiment, the MMAD of the particles is between about 5 μm and about 10 μm. In one embodiment, the MMAD of the particles is between about 0.5 μm and about 5 μm.

The particles may be administered to the lungs by any route. In some embodiments, the particles are inhaled into the lung. In one embodiment, the particles are administered by a nebulizer. In one embodiment, the particles are administered by a metered-dose inhaler. In one embodiment, the particles are administered by a dry powder inhaler. Materials for use in each type of device are well-known in the art and can be readily determined by the skilled clinician.

Where the composition is in a formulation to be applied topically, e.g., directly to tissues, e.g., in the peritoneal cavity, it may be in the form of a gel, cream, lotion, or the like. In some embodiments, the composition is in a sprayable formulation. Such formulations are well known in the art.

In one aspect, the composition can include one or more surfactants and/or emulsifiers. Surfactants (or surface-active substances) that may be present are anionic, non-ionic, cationic and/or amphoteric surfactants. Typical examples of anionic surfactants include, but are not limited to, soaps, alkylbenzenesulfonates, alkanesulfonates, olefin sulfonates, alkyl ether sulfonates, glycerol ether sulfonates, α-methyl ester sulfonates, sulfo fatty acids, alkyl sulphates, fatty alcohol ether sulphates, glycerol ether sulphates, fatty acid ether sulphates, hydroxy mixed ether sulphates, monoglyceride (ether) sulphates, fatty acid amide (ether) sulphates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, e.g., acyl lactylates, acyl tartrates, acyl glutamates and acyl aspartates, alkyl oligoglucoside sulphates, protein fatty acid condensates (in particular wheat-based vegetable products) and alkyl (ether) phosphates. Examples of non-ionic surfactants include, but are not limited to, fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. Examples of amphoteric or zwitterionic surfactants include, but are not limited to, betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethyl-carboxymethyl glycinate, alkylbetaines, alkylamidobetaines, aminopropionates, aminoglycinates, imidazolinium-betaines and sulfobetaines.

In some embodiments, the surfactant can be fatty alcohol polyglycol ether sulphates, monoglyceride sulphates, mono- and/or dialkyl sulfosuccinates, fatty acid isethionates, fatty acid sarcosinates, fatty acid taurides, fatty acid glutamates, alpha-olefinsulfonates, ether carboxylic acids, alkyl oligoglucosides, fatty acid glucamides, alkylamidobetaines, amphoacetals and/or protein fatty acid condensates.

In some embodiments, the emulsifier can be a nonionogenic surfactant selected from the following: the addition products of from 2 to 30 mole of ethylene oxide and/or 0 to 5 mole of propylene oxide onto linear fatty alcohols having 8 to 22 carbon atoms, onto fatty acids having 12 to 22 carbon atoms, onto alkylphenols having 8 to 15 carbon atoms in the alkyl group, or onto alkylamines having 8 to 22 carbon atoms in the alkyl radical; alkyl and/or alkenyl oligoglycosides having 8 to 22 carbon atoms in the alk(en)yl radical and the ethoxylated analogs thereof; the addition products of from 1 to 15 mole of ethylene oxide onto castor oil and/or hydrogenated castor oil; the addition products of from 15 to 60 mole of ethylene oxide onto castor oil and/or hydrogenated castor oil; partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mole of ethylene oxide; partial esters of polyglycerol (average degree of self-condensation 2 to 8), trimethylolpropane, pentaerythritol, sugar alcohols (e.g., sorbitol), alkyl glucosides (e.g., methyl glucoside, butyl glucoside, lauryl glucoside), and polyglucosides (e.g., cellulose) with saturated and/or unsaturated, linear or branched fatty acids having 12 to 22 carbon atoms and/or hydroxycarboxylic acids having 3 to 18 carbon atoms, and the adducts thereof with 1 to 30 mole of ethylene oxide; mixed esters of pentaerythritol, fatty acids, citric acid and fatty alcohols and/or mixed esters of fatty acids having 6 to 22 carbon atoms, methylglucose and polyols, for example glycerol or polyglycerol, mono-, di- and trialkyl phosphates, and mono-, di- and/or tri-PEG alkyl phosphates and salts thereof; wool wax alcohols; polysiloxane-polyalkyl-polyether copolymers and corresponding derivatives; and block copolymers, e.g., polyethylene glycol-30 dipolyhydroxystearates.

In some embodiments, the emulsifier is a polyalkylene glycol such as, for example, polyethylene glycol or polypropylene glycol. In some embodiments, the emulsifier is polyethylene glycol having a molecular weight 100 Da to 5,000 Da, 200 Da to 2,500 Da, 300 Da to 1,000 Da, 400 Da to 750 Da, 550 Da to 650 Da, or about 600 Da.

In some embodiments, the emulsifier is a poloxamer.

In some embodiments, the emulsifier is composed of one or more fatty alcohols. In some embodiments, the fatty alcohol is a linear or branched C₆ to C₃₅ fatty alcohol. Examples of fatty alcohols include, but are not limited to, capryl alcohol (1-octanol), 2-ethyl hexanol, pelargonic alcohol (1-nonanol), capric alcohol (1-decanol, decyl alcohol), undecyl alcohol (1-undecanol, undecanol, hendecanol), lauryl alcohol (dodecanol, 1-dodecanol), tridecyl alcohol (1-tridecanol, tridecanol, isotridecanol), myristyl alcohol (1-tetradecanol), pentadecyl alcohol (1-pentadecanol, pentadecanol), cetyl alcohol (1-hexadecanol), palmitoleyl alcohol (cis-9-hexadecen-1-ol), heptadecyl alcohol (1-n-heptadecanol, heptadecanol), stearyl alcohol (1-octadecanol), isostearyl alcohol (16-methylheptadecan-1-ol), elaidyl alcohol (9E-octadecen-1-ol), oleyl alcohol (cis-9-octadecen-1-ol), linoleyl alcohol (9Z,12Z-octadecadien-1-ol), elaidolinoleyl alcohol (9E,12E-octadecadien-1-ol), linolenyl alcohol (9Z,12Z,15Z-octadecatrien-1-ol) elaidolinolenyl alcohol (9E,12E,15-E-octadecatrien-1-ol), ricinoleyl alcohol (12-hydroxy-9-octadecen-1-ol), nonadecyl alcohol (1-nonadecanol), arachidyl alcohol (1-eicosanol), heneicosyl alcohol (1-heneicosanol), behenyl alcohol (1-docosanol), erucyl alcohol (cis-13-docosen-1-ol), lignoceryl alcohol (1-tetracosanol), ceryl alcohol (1-hexacosanol), montanyl alcohol, cluytyl alcohol (1-octacosanol), myricyl alcohol, melissyl alcohol (1-triacontanol), geddyl alcohol (1-tetratriacontanol), or cetearyl alcohol.

In some embodiments, a carrier is used to produce the composition. In some embodiments, the carrier is a mixture of polyethylene and one or more fatty alcohols. For example, the carrier comprises about 50% to about 99% by weight, about 75% to about 99% by weight, about 90% to about 99% by weight, or about 95% by weight polyethylene glycol and about 1% to about 50% by weight, about 1% to about 25% by weight, about 1% to about 10% by weight, or about 5% by weight fatty alcohol. In some embodiments, the carrier is a mixture of polyethylene glycol and cetyl alcohol.

In some embodiments, the composition can include one or more of the following components: fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, swelling agents, insect repellents, hydrotropes, solubilizers, preservatives, perfume oils and dyes. Examples of each of these components are disclosed in U.S. Pat. No. 8,067,044, which is incorporated by reference with respect these components.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as embodiments, and are not intended to be limiting.

### EXAMPLES

The following examples are for illustrative purposes only and should not be interpreted as limitations of the claimed invention. There are a variety of alternative techniques and procedures available to those of skill in the art, which would similarly permit one to successfully perform the intended invention.

### Example 1

### Effective of CXCL12 Incorporation on Implanted Microbeads

CXCL12 preparation with or without coating on implanted cells, tissues, organs or device to reduce or abrogate the formation of adhesions and the other sequelae of intra-body cavity inflammation including peritonitis, pericarditis and pleuritis. CXCL12 could also be directly applied to these body cavities when inflamed due to auto-immune diseases associated with serositis (including inflammation of the peritoneal mesothelium, pericardial membrane or pleural membrane inflammation).

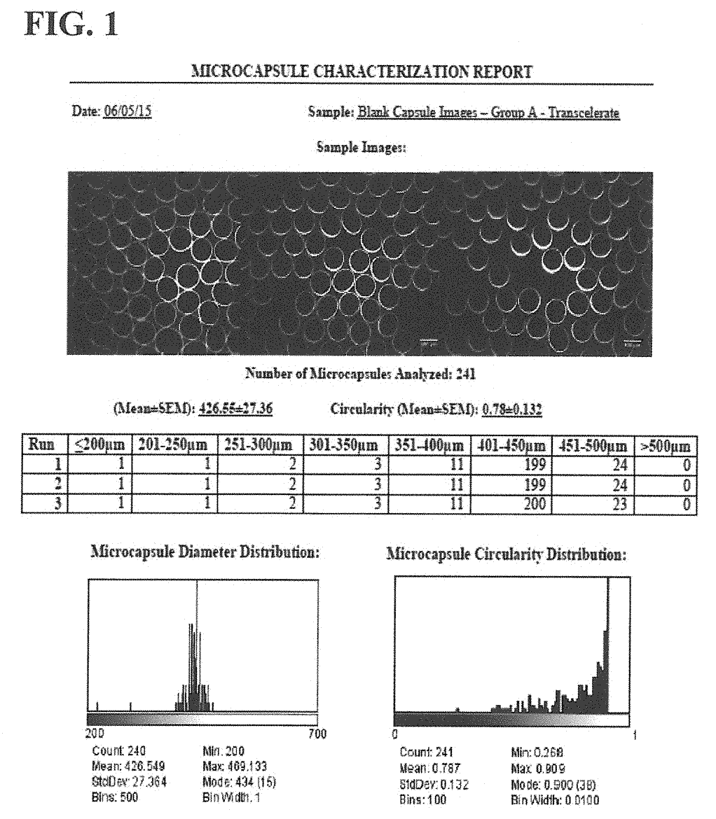

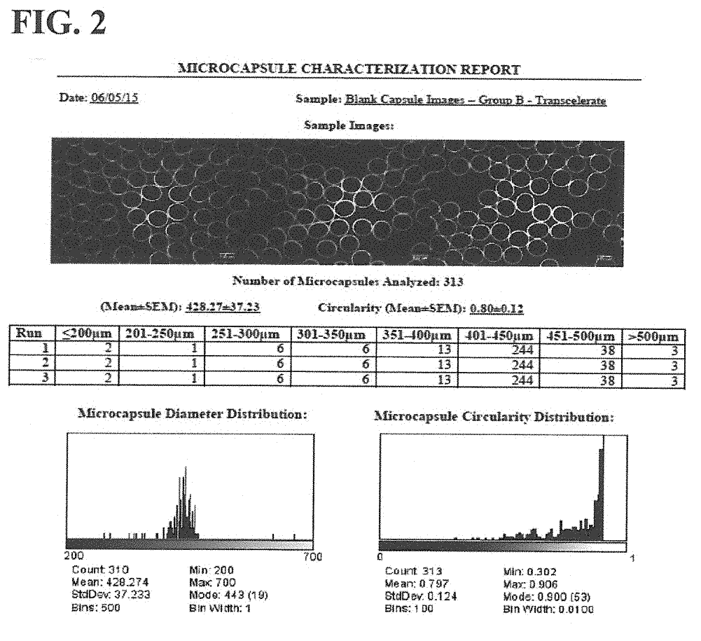

In the first non-human primate (NHP) study with alginate microbeads, two animals received intraperitoneal implants of ˜400,000 microbeads each of (1.6% calcium LVM) alginate containing either 1 μg/ml of recombinant human CXCL12 (“CXCL12⁺”) or not (“CXCL12”). Microbeads were prepared using a vibrational encapsulation method using a 200 μm nozzle. Microbeads were on average 426-427 μm in diameter with an error of the mean of approximately 10-15%. The diameter was measured after 2 hours in culture media. Microbeads swell 30-40% after transfer into some media or after implantation. Median roundness was less than the desirable 0.90, ranging from 0.78 in the CXCL12⁻ microbeads (FIG. 1) to 0.80 in the CXCL12⁺ (FIG. 2), but with a skew in roundness reflecting a small but significant number of non-round beads.

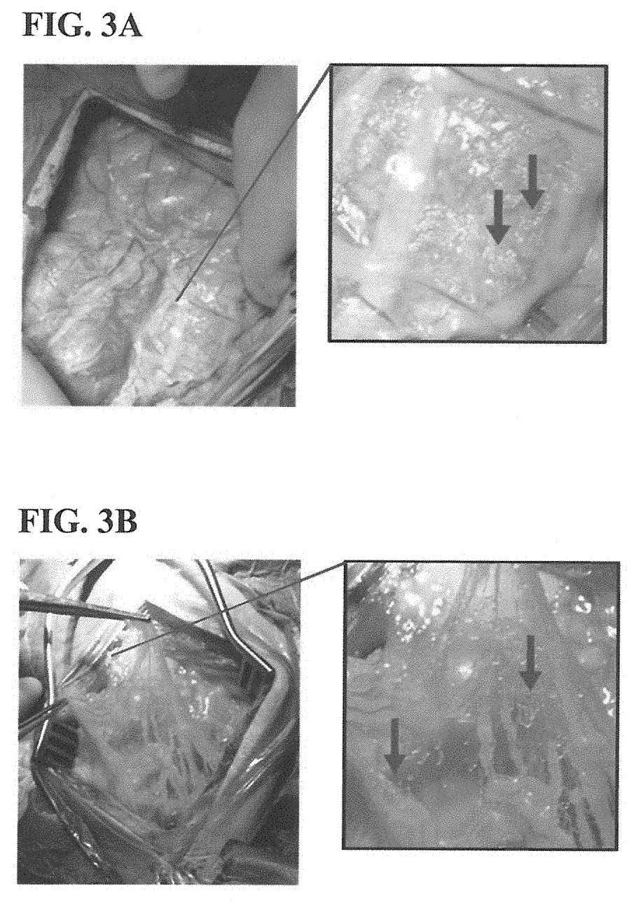

Microbeads were delivered in a blinded manner to operators. During the implant, primate 0715 received the CXCL12⁻ implant; primate 1715 received the CXCL12⁺ implant. Both animals were implanted using a minimally invasive technique featuring placement of beads in 30 cc of 300 mOsmolar calcium chloride solution in the intraperitoneal space and explanted surgically. Neither of the procedures was eventful. Both animals appeared healthy during the entire period of four weeks following the implantation procedure. The explant procedure for each animal involved surgical opening of the peritoneal cavity, visual inspection of all regions of the mesentery and abdomen/pelvis for bead position, lavage with 100 cc of N-saline to recover free microbeads and peritoneal fluid, and biopsy of tissues where microbeads appeared to be embedded in the tissue. Evaluation was done at 30, 90, and 180 days. Images from the explant procedure at 30 days are shown in FIGS. 3A-3B. Arrows point to adhesions.

Upon visual inspection, microbeads were widely distributed without specific anatomic site of retention or accumulation. No intra-abdominal inflammation or physical evidence of adhesions were apparent in either NHP. Islet microbeads appeared distributed throughout the peritoneum in both animals. While some microbeads were found in the recto-vesical pouch, this was not the predominant location. There was minimal embedding of microbeads in the intraperitoneal tissue.

At later timepoints, the long-term appearance of the peritoneum was more normal and without surgical adhesions in the CXCL12⁺ NHP compared to the CXCL12⁻ NHP. The CXCL12⁻ NHP developed patchy fibrosis and had surgical adhesions starting at day 90 and clearly evident at day 180. The microbeads lightly adhered to tissue surfaces. They remained scatter throughout the abdominal cavity with no significant concentration in the pelvic floor. The majority of CXCL12+ microbeads were recoverable by saline lavage (70% recovered by the end of six months).

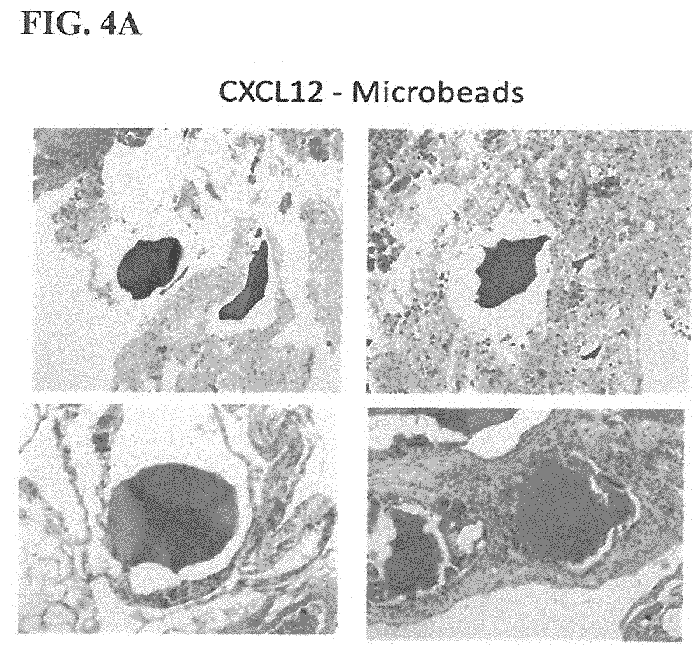

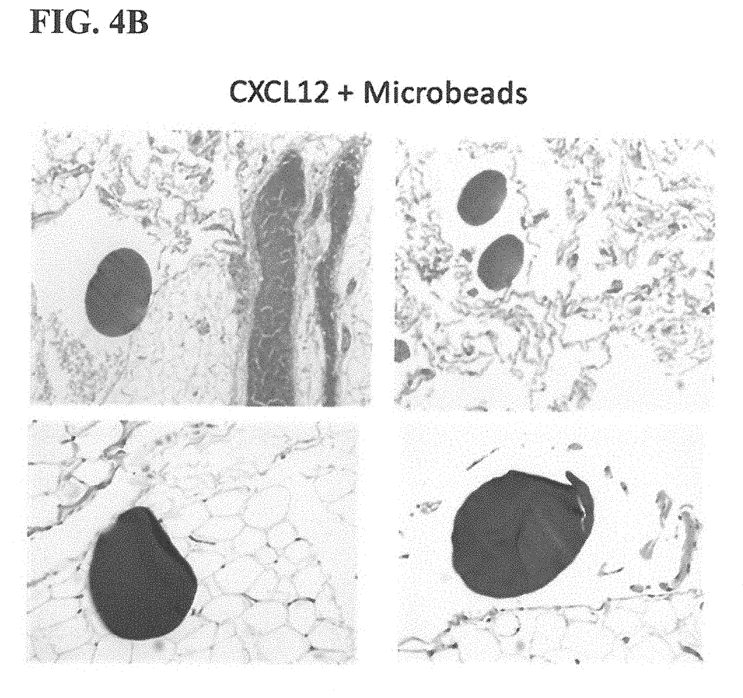

The histopathology of biopsied tissue at 30 days was examined. H&E stained sections are shown in FIGS. 4A and 4B. Fibrosis was present in CXCL12⁻ microbeads but not in CXCL12⁺ microbeads.

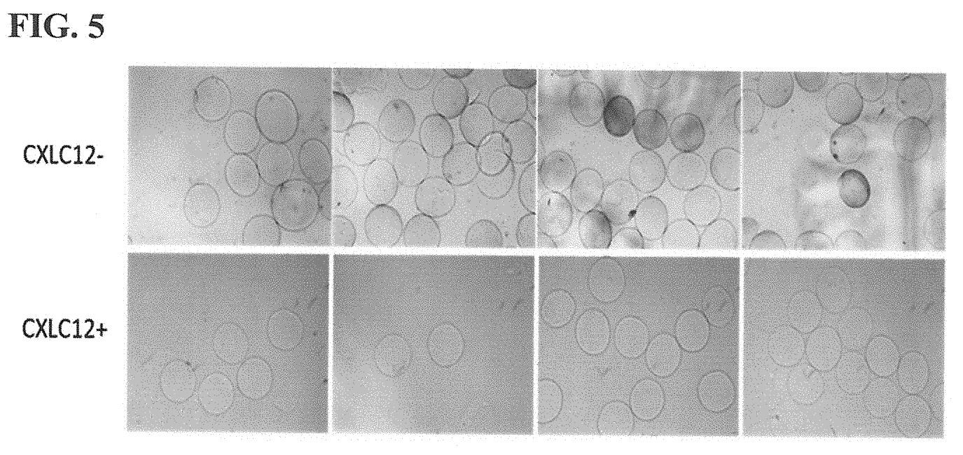

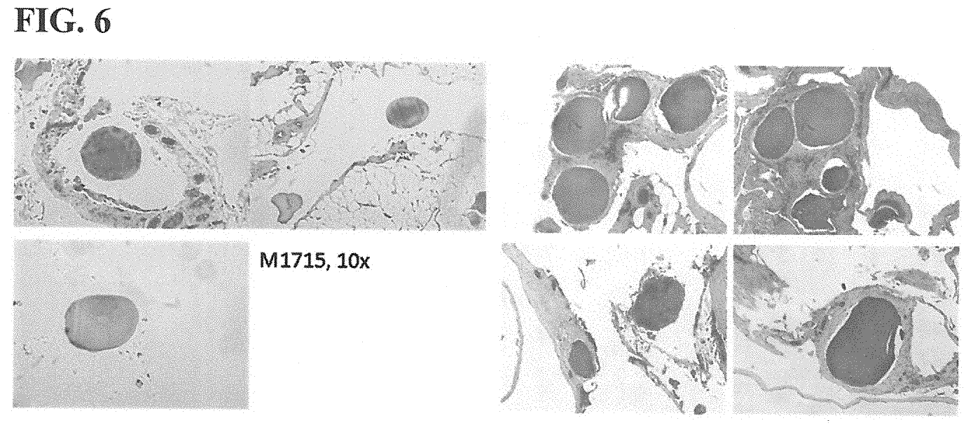

CXCL12 significantly improved the long-term condition of free microbeads. The CXCL12⁺ microbeads showed reduced irregularity, cellularization, and discoloration starting at 90 days and continuing through 180 days compared to CXCL12− microbeads (FIG. 5). CXCL12 appeared to spare embedded microbeads from foreign body responses through 90 days. H&E stained sections from 90 day and 180 day biopsies are shown in FIG. 6, with fibrosis evident at 90 days with CXCL12⁻ microbeads.

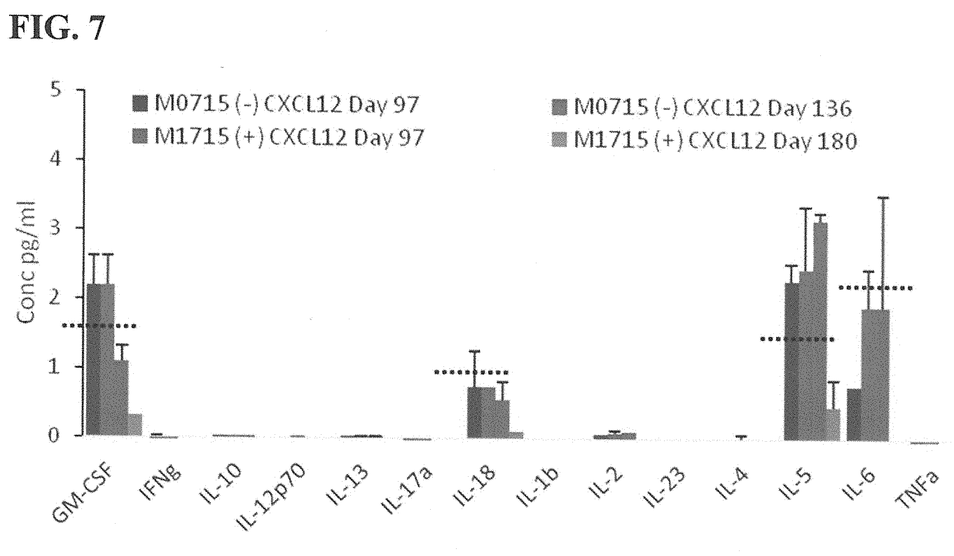

Cytokine levels were measured in the plasma of both animals. There was no significant difference between NPs at 90 days but cytokine levels in the CXCL12+ animal were lower at 180 days (FIG. 7).

In summary, the CXCL12⁺ microbeads evoked no histopathologically evident fibrosis up to 90 days post intraperitoneal insertion and minimal fibrosis at 180 days post implant. There was no surgical evidence of intraabdominal adhesions to 180 days post implantation. In contrast, the CXCL12⁻ microbeads evoked histopathologically evident fibrosis from 30 days post intraperitoneal insertion and significant fibrosis beyond that time point to 180 days post implant. There was clear surgical evidence of intraabdominal surgical adhesions from 90 days and again at 180 days post implantation. Thus, CXCL12 appears to retard fibrosis and abrogate adhesion formation following surgery in the intraperitoneal cavity.

### Example 2

### Xenoislets Containing Microbeads with CXCL12

Xenoislets containing microbeads ±CXCL12 were prepared as follows. 100,000+ islet equivalents (IEQ) of adult porcine islets were obtained. The islets were placed in recovery culture for 1-2 days in G-Rex flasks with Prodo Complete Media preparation plus antibiotics. The islets were encapsulated using an air flow-based encapsulation technique using about 100,000 IEQ islets at a ratio of about 1.5 beads per IEQ with the beads having a mean diameter of 600-650 μm (±5%) and roundness above 0.8±1 μg/ml CXCL12. The encapsulated islets were placed in recovery culture for 1-2 days in G-Rex flasks with Prodo media plus inactivated cyno serum plus antibiotics. The final product had a functional viability above or equal to the shipped islets and endotoxin levels <1 EU/ml alginate. The encapsulated islets were implanted into the natural omental pouch of diabetic NHPs. Blood/serum assays were parted at 0, 1, 2, 3, 4, 13, and 26 weeks. Surgical assessment was done at days 30 and 90 (or euthanasia end point).

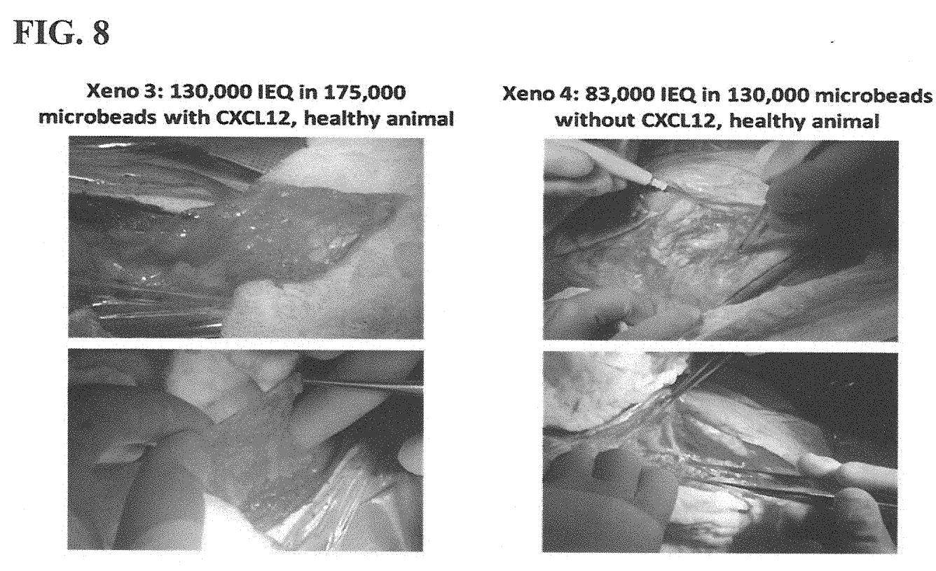

In the CXCL12⁻ xenoislet implant significant and massive fibrotic adhesions (arrows) were present which largely destroyed the graft by 30 days post implantation (FIG. 8). In contrast, the CXCL12⁺ xenoislets showed an absence of any fibrotic intraperitoneal adhesions (FIG. 8).

The foregoing is illustrative of the present invention, and is not to be construed as limiting thereof. The invention is defined by the following claims, with equivalents of the claims to be included therein.

1. A method for preventing or attenuating fibrosis and/or adhesion formation in a subject in need thereof, the method comprising administering to the subject an effective amount of a composition comprising a CXCR4 and/or CXCR7 binding agent, thereby preventing or attenuating fibrosis and/or adhesion formation.

2. The method of claim 1, wherein the composition is administered during or immediately after surgery.

3. The method of claim 2, wherein the surgery is intra-abdominal surgery or intra-thoracic surgery.

4. The method of claim 1, wherein the composition is administered during or immediately after insertion of a device or implant into the subject.

5. The method of any one of claim 1, wherein the composition comprises particles loaded with the CXCR4 and/or CXCR7 binding agent.

6. The method of claim 5, wherein the particles are immunorepellant, biodegradable, and non-cellular particles.

7. The method of claim 5, wherein the particles encapsulate or are coated with the CXCR4 and/or CXCR7 binding agent.

8. The method of claim 5, wherein the CXCR4 and/or CXCR7 binding agent elutes from the particles in an amount sufficient to provide a chemorepellant environment at the site of administration for a period of at least one month after administration.

9. The method of claim 1, wherein the composition is a sprayable composition.

10. The method of claim 1, wherein the composition is a topical composition.

11. The method of claim 1, wherein the CXCR4 and/or CXCR7 binding agent is CXCL12.

12. A method for preventing or attenuating scarring in a subject in need thereof, the method comprising administering to the subject a composition comprising an effective amount of a CXCR4 and/or CXCR7 binding agent to a site of potential scarring, thereby preventing or attenuating scarring.

13. The method of claim 12, wherein the scarring is due to surgery.

14. The method of claim 13, wherein abdominal adhesions are prevented or attenuated.

15. The method of claim 12, wherein the composition is administered during or immediately after surgery.

16. The method of claim 12, wherein the surgery is intra-abdominal surgery or intra-thoracic surgery.

17. The method of claim 12, wherein the composition comprises particles loaded with the CXCR4 and/or CXCR7 binding agent.

18-22. (canceled)

23. The method of claim 12, wherein the CXCR4 and/or CXCR7 binding agent is CXCL12.

24. A method for treating, preventing, or inhibiting progression of a fibrotic disease in a subject in need thereof, the method comprising administering to the subject a composition comprising an effective amount of a CXCR4 and/or CXCR7 binding agent to the site of fibrosis, thereby treating, preventing, or inhibiting progression of the fibrotic disease.

25. (canceled)

26. The method of claim 24, wherein the fibrotic disease is fibrotic lung disease.

27-28. (canceled)

29. The method of claim 24, wherein the CXCR4 and/or CXCR7 binding agent is CXCL12.

30. (canceled)

31. The method of claim 24, wherein the composition comprises particles loaded with the CXCR4 and/or CXCR7 binding agent.

32-44. (canceled)
